(19) United States
(12) Patent Application Publication
(10) Pub. No.: US 2021/0383905 A1
(43) Pub. Date: Dec. 9, 2021

(54) MEDICAL INFORMATION PROCESSING APPARATUS, MEDICAL INFORMATION PROCESSING SYSTEM, MEDICAL INFORMATION PROCESSING METHOD, AND STORAGE MEDIUM

(71) Applicants: Canon Kabushiki Kaisha, Tokyo (JP); CANON MEDICAL SYSTEMS CORPORATION, Tochigi (JP)

(72) Inventor: Toru Kikuchi, Tokyo (JP)

(21) Appl. No.: 17/335,514

(22) Filed: Jun. 1, 2021

(30) Foreign Application Priority Data
Jun. 3, 2020 (JP) 2020-097140

(51) Int. Cl. G16H 10/60; G06T 7/00; G16H 30/20

(52) U.S. Cl. CPC G16H 10/60; G06T 2207/30096; G16H 30/20; G06T 7/0012

(57) ABSTRACT

A medical information processing apparatus comprises: an obtaining unit configured to obtain a plurality of findings related to a lesion to be diagnosed; a selection unit configured to select a finding based on a predetermined condition from the plurality of findings; a report generation unit configured to generate, based on content of the selected finding, a report relating to the lesion; a setting unit configured to set whether or not to maintain a selected state of the finding selected by the selection unit; and an updating unit configured to update the report based on content of a finding selected in accordance with a setting of whether or not to maintain the selected state set by the setting unit.

### BACKGROUND OF THE INVENTION

### Field of the Invention

The present invention relates to a medical information processing apparatus, a medical information processing system, a medical information processing method, and a storage medium.

### Description of the Related Art

A Computer Aided Diagnosis (CAD) system that analyzes medical images and the like with a computer and presents information which supports a diagnosis to a doctor is known.

In Japanese Patent Laid-Open No. 2009-516551 (PTL1), a CAD system that analyzes a plurality of medical image data, which relates to an area within an anatomical tissue, to extract a feature, associates the extracted feature between the plurality of medical image data, and calculates an overall evaluation of a possibility of a lesion is disclosed. The CAD system of PTL1 presents to a user an extracted feature or calculated overall evaluation by an analysis of a plurality of medical image data, and the user can confirm the presented feature or overall evaluation and perform a correction. The CAD system of PTL1 generates an interpretation report by using the feature or overall evaluation confirmed and corrected by the user, and when the user has corrected the feature, can recalculate the overall evaluation by using the corrected feature.

Also, in Japanese Patent Laid-Open No. 2013-167977 (PTL2), a medical diagnosis support apparatus that obtains, as input information, imaging findings and the like obtained from a medical image, and after inferring a diagnostic name based on the obtained input information, calculates a degree of influence of the input information on each inference, selects the input information based on the calculated influence degree, and generates a report is disclosed.

Depending on a case to be evaluated, the number of findings required for diagnosing the case is large, and a case can arise in which a report becomes complicated when all the findings are described in the report.

In the CAD system described in PTL1, it is not possible to select a finding and generate a report. Also, in the medical diagnosis support apparatus described in PTL2, although it is possible to automatically select a finding based on a degree of influence and generate a report, it is not possible for a user to set a finding to be described in a report or a finding to not be described in a report.

In consideration of the abovementioned problems, the present invention provides a medical information processing technique in which a user can set a finding to be described or to not be described in a report as a setting of a selected state of a finding, and in which it is possible to update the report based on contents of findings selected in accordance with the setting of the selected state when the report is to be updated.

### SUMMARY OF THE INVENTION

According to one aspect of the present invention, there is provided a medical information processing apparatus comprising: an obtaining unit configured to obtain a plurality of findings related to a lesion to be diagnosed; a selection unit configured to select a finding based on a predetermined condition from the plurality of findings; a report generation unit configured to generate, based on content of the selected finding, a report relating to the lesion; a setting unit configured to set whether or not to maintain a selected state of the finding selected by the selection unit; and an updating unit configured to update the report based on content of a finding selected in accordance with a setting of whether or not to maintain the selected state set by the setting unit.

Further features of the present invention will become apparent from the following description of exemplary embodiments (with reference to the attached. drawings).

### BRIEF DESCRIPTION OF THE DRAWINGS

FIG. 1 is a view illustrating a configuration of a medical information processing system according to a first or second embodiment.

FIG. 2 is a view illustrating a hardware configuration of a medical information processing apparatus according to the first or second embodiment.

FIG. 3 is a view illustrating a functional configuration of function of the medical information processing apparatus according to the first embodiment.

FIG. 4 is a view illustrating an example of a user interface screen of the medical information processing apparatus according to the first embodiment.

FIG. 5 is a flowchart illustrating processing of the medical information processing apparatus according to the first embodiment.

FIG. 6 is a view illustrating a functional configuration of function of the medical information processing apparatus according to the second embodiment.

FIG. 7 is a view illustrating an example of the user interface screen of the medical information processing apparatus according to the second embodiment.

FIG. 8 is a flowchart illustrating processing of the medical information processing apparatus according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

In the first embodiment, the medical information processing apparatus which supports generation of an interpretation report with respect to a shadow of a pulmonary nodule on an image of a chest X-ray CT (Computed Tomography) is explained.

The medical information processing apparatus in the present embodiment firstly estimates an imaging finding from an image of a shadow of a pulmonary nodule. The imaging finding is information for indicating characteristics of a pulmonary nodule such as “overall shape”, “boundary”, and “edge” of a pulmonary nodule.

The imaging finding relating to “overall shape” is given a value such as “spherical shape”, “lobed shape”, “polygonal shape”, “wedge shape”, “flat shape”, and “irregular shape”. An imaging finding relating to “boundary” is given a value such as “clear” or “unclear”, and an imaging finding relating to “edge” is given a value such as “ordered” or “disordered”. Hereinafter, types of imaging findings such as “overall shape”, “boundary”, and “edge” are described and values or contents of imaging findings such as “spherical shape”, “clear”, and “ordered” are described. For an estimation of an imaging finding, a known Convolutional Neural Network (CNN) trained by supervisory data may be used.

The medical information processing apparatus of the present embodiment estimates a diagnostic name by using the estimated imaging finding. The diagnostic name is, for example, “benign pulmonary nodule”, “primary lung cancer”, “metastatic lung cancer”, and the like. For an estimation of the diagnostic name, a known Bayesian Network trained by supervisory data may be used. Also, when the medical information processing apparatus estimates the diagnostic name, it calculates a degree of influence of each imaging finding inputted with respect to the estimated diagnostic name. Next, the medical information processing apparatus selects an imaging finding in which the degree of influence exceeds a predetermined threshold, and automatically generates a report by applying the selected imaging finding and diagnostic name to a template (hereinafter referred to as “an interpretation report”).

### Configuration of a Medical Information Processing System

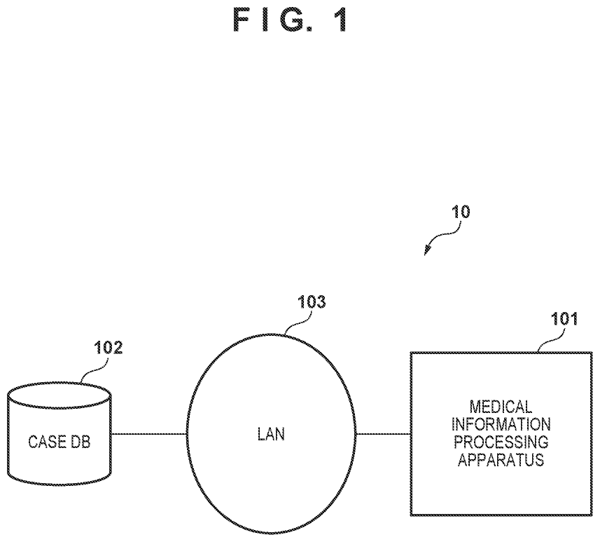

FIG. 1 is a view illustrating a configuration of a medical information processing system 10 which includes the medical information processing apparatus of the present embodiment. As illustrated in FIG. 1, the medical information processing system 10 has a case database (hereinafter referred to as “a case DB”) 102, a medical information processing apparatus 101, and a Local Area Network (LAN) 103.

The case DB 102 functions as a storage unit for storing medical images captured by an apparatus that captures medical images such as an X-ray CT apparatus and medical information to be attached to the medical images. Coordinate information which indicates a position and range of a lesion within a medical image is also included in the medical information. The medical information to be attached to the medical image may be stored as header information of the medical image. The case DB 102 can provide a database function and the medical information processing apparatus 101 can search for medical images of the case DB 102 by using the medical information and obtain medical images from the case DB 102 via the LAN 103.

### Hardware Configuration

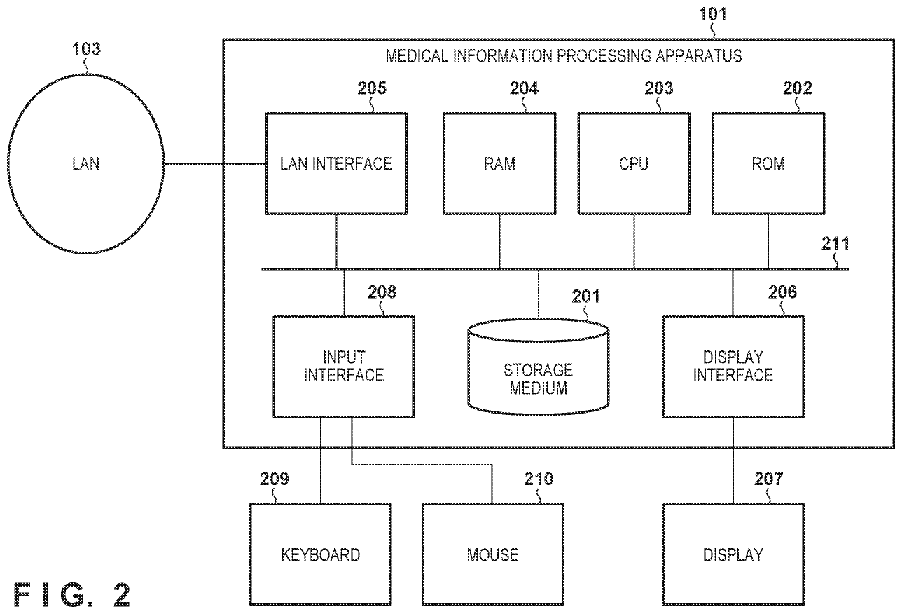

FIG. 2 is a view illustrating a hardware configuration of the medical information processing apparatus 101 of the present embodiment. In FIG. 2, the medical information processing apparatus 101 has a storage medium 201, a Read Only Memory (ROM) 202, a Central Processing Unit (CPU) 203, and a Random Access Memory (RAM) 204. Furthermore, the medical information processing apparatus 101 has a LAN interface 205, an input interface 208, a display interface 206, and an internal bus 211.

The storage medium 201 is a storage medium such as a Hard Disk Drive (HDD) that stores an Operating System (OS), a processing program for performing various processing according to the present embodiment, and various information. The ROM 202 stores a Basic Input Output System (BIOS) and the like and a program for initializing hardware and activating the OS. The CPU 203 performs computation processing when executing the BIOS, OS, or processing program. The RAM 204 temporarily stores information when the CPU 203 executes a program. The LAN interface 205 is an interface for performing communication via the LAN 103 and complies with a standard such as IEEE (Institute of Electrical and Electronics Engineers) 802.3ab.

A display 207 displays a display screen, the display interface 206 converts the image information to be displayed on the display 207 to a signal, and the display 207 outputs the result. The CPU 203 and the display interface 206 function as display control units for controlling display of the display 207. A keyboard 209 performs key inputs, a mouse 210 designates a coordinate position on the screen and performs input of button operations, and the input interface 208 receives the signal inputted from the keyboard 209 and the mouse 210. The internal bus 211 performs transmission of a signal when communication is performed between each block of the functional configuration.

### Functional Block

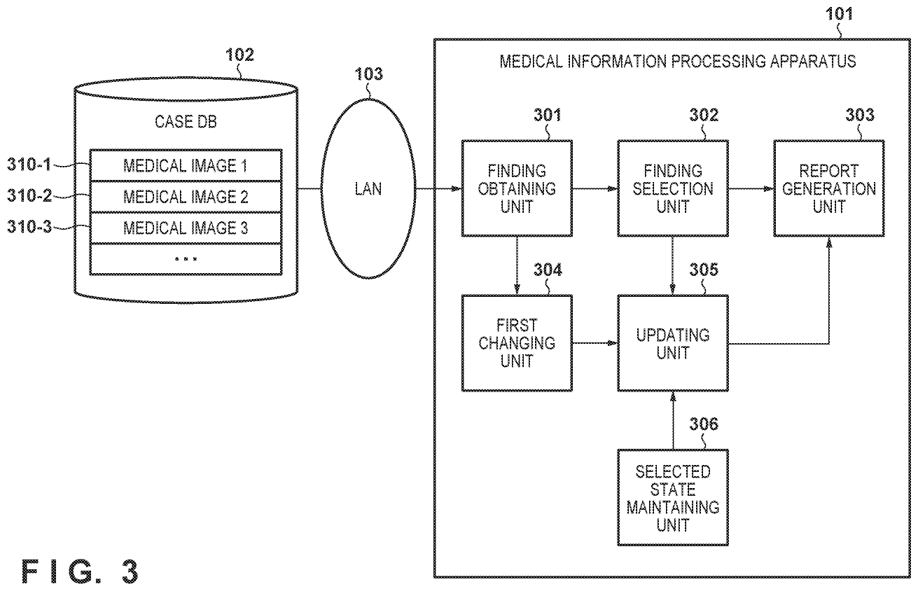

FIG. 3 is a view illustrating functional blocks of the medical information processing apparatus 101 of the present embodiment. In FIG. 3, the medical information processing apparatus 101 has a finding obtaining unit 301, a finding selection unit 302, a report generation unit 303, a first changing unit 304, an updating unit 305, and a selected state maintaining unit 306 as functional blocks. A plurality of medical images 310-i (i=1, 2, 3, . . . ) are saved in the case DB 102. These functional blocks can be realized by the CPU 203, which functions as a control unit of the medical information processing apparatus 101, executing a processing program read from the storage medium 201. The configuration of each functional block may be formed by an integrated circuit or the like as long as a similar function can be achieved.

The finding obtaining unit 301 obtains a plurality of findings related to a lesion to be diagnosed. The finding obtaining unit 301 obtains an imaging finding with respect to lesions of the medical images 310-i to be diagnosed. Specifically, the medical images 310-i to be diagnosed are obtained via the LAN 103 (network) from the case DB 102 and imaging findings are obtained by analyzing the medical images 310-i. The finding obtaining unit 301 can use a CNN for analysis of the imaging findings, and one CNN may estimate one imaging finding or a plurality of imaging findings. The finding obtaining unit 301 obtains the medical images 310-i and medical information to be attached to the medical images. Coordinate information indicating a position and/or range of a lesion within a medical image is also included in the medical information, and the finding obtaining unit 301, by obtaining information relating to the position and range of the lesion from the medical information and, based on the position and range of the obtained lesion, performing analysis using a part of the medical images 310-i as an input, may estimate the imaging findings.

The finding selection unit 302 selects a finding based on a predetermined condition from a plurality of findings. For example, the finding selection unit 302 calculates a degree of influence of each finding with respect to the diagnostic name estimated from the plurality of findings and selects a finding for which the degree of influence is greater than a preset value. Firstly, the finding selection unit 302 selects an imaging finding used for generation of a report from the imaging findings obtained by the finding obtaining unit 301. In the selection of the imaging finding, the finding selection unit 302 first estimates a diagnostic name from the imaging findings. Next, the finding selection unit 302 calculates a degree of influence of each imaging finding with respect to the estimated diagnostic name. Finally, the finding selection unit 302 selects an imaging finding for which the calculated degree of influence is greater than or equal to a preset value as an imaging finding to be used in a report. Here, the finding selection unit 302 can use a Bayesian Network for estimation of a diagnostic name. Here, the degree of influence is a difference between a likelihood of a diagnosis estimation result for which an imaging finding is not inputted and a likelihood of a diagnosis estimation result for which imaging findings have been individually inputted.

The report generation unit 303 generates a report relating to a lesion based on the content of the selected finding. The report generation unit 303 generates text of a report by applying the imaging finding selected by the finding selection unit 302 to a template. For example, when the imaging finding such as {X}=“irregular shape”, {Y}=“spicule”, “sawtooth edge” is applied to a template such as “A node of {X} is recognized in the right lung. Accompanied by {Y}.”, the report generation unit 303 generates text of a report saying “An irregular shape node of the right lung is observed. Accompanied by spicula and a sawtooth edge”. Note, the above described template and imaging finding are an example and the present invention is not limited to this.

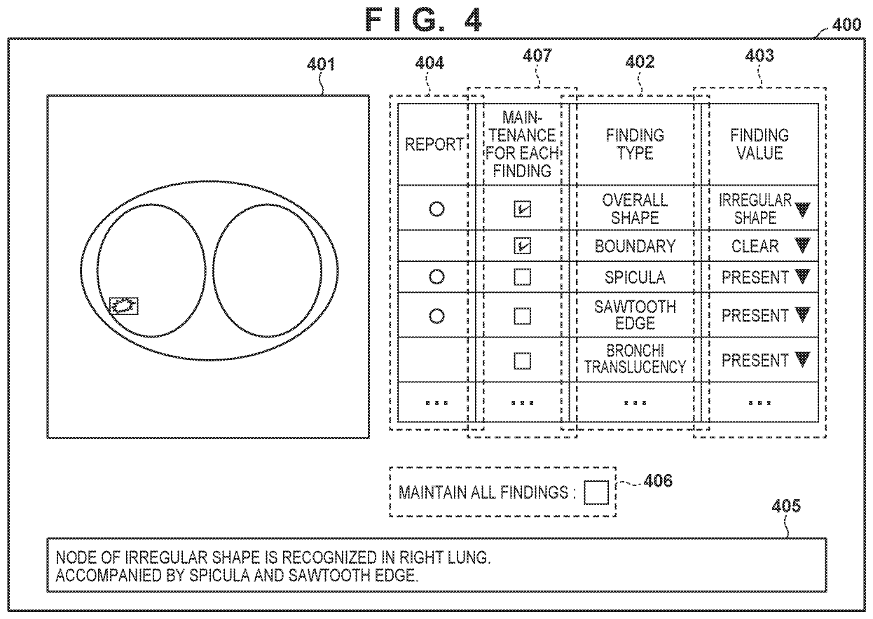

The first changing unit 304 changes content of at least one finding among a plurality of findings based on an instruction (first instruction) from an input unit such as the keyboard 209 or the mouse 210 as an operation input from the user. The first changing unit 304 changes the content (value) of the imaging finding obtained by the finding obtaining unit 301. For example, when the value of the imaging finding, which was obtained by the finding obtaining unit 301, relating to “overall shape” as a type is “irregular shape”, the first changing unit 304 changes the value to another value such as “spherical shape”, “lobed shape”, or “polygonal shape” based on the operation input. A change based on the operation input is carried out in accordance with an operation of the user interface screen, such as a pull-down menu or a radio button, for example. Description regarding the user interface screen is given using FIG. 4.

The updating unit 305 receives a change of values of imaging findings by the first changing unit 304 and regenerates and updates a report by using the imaging findings that were changed and the imaging findings that were not changed. Here, the imaging findings to be used in the report are reselected by using the finding selection unit 302 and the report is regenerated and updated by using the updating unit 305. The updating unit 305 updates the report based on the contents of the findings selected according to the setting of the selected state in accordance with the change of the values of the imaging findings. In other words, the updating unit 305 updates the report that had already been generated in the report generation unit 303 using the regenerated report.

The selected state maintaining unit 306 sets whether or not to maintain the selected state of the findings selected by the finding selection unit 302. When a report is updated by the updating unit 305, the selected state maintaining unit 306 operates so as to maintain the selected states based on a designation of whether or not the imaging findings are to be used in the regeneration of the report. The updating unit 305 updates the generated report based on the contents of the findings selected by the finding selection unit 302 in accordance with the setting of whether or not to maintain the selected states set by the selected state maintaining unit 306.

The finding selection unit 302 selects findings according to the setting of the selected state maintaining unit 306 when it reselects at least one finding among the plurality of findings in accordance with the change of the content of the finding. The finding selection unit 302 selects contents of the findings selected in accordance with the setting of whether or not to maintain the selected states set by the selected state maintaining unit 306. Among the imaging findings selected by the finding selection unit 302, regarding the imaging finding designated, the selected state maintaining unit 306 maintains the selected states, and regarding the imaging finding not designated, the selected state maintaining unit 306 clears the selected states. The designation of the imaging findings which maintain the selected states is set based on the designation by the user interface screen such as a checkbox or a button. Description regarding the user interface screen is given using FIG. 4.

The updating unit 305 updates the report based on the contents of the findings selected in accordance with the setting of whether or not to maintain the selected states set by the selected state maintaining unit 306 and the changes in the first changing unit 304. In other words, the updating unit 305 updates the report that had already been generated by the report generation unit 303 using the regenerated report.

### User Interface Screen

FIG. 4 is a view illustrating an example of a user interface screen 400 of the medical information processing apparatus 101 according to the present embodiment. The user interface screen 400 is displayed on the display 207, and an operation on the user interface screen 400 is performed by the keyboard 209 or the mouse 210. The CPU 203 and the display interface 206 function as display control units and control display of the display 207.

In FIG. 4, the user interface screen 400 has a medical image displaying area 401, a finding type displaying area 402, a finding value displaying area 403, a selected state displaying area 404, and a report displaying area 405. Furthermore, the user interface screen 400 has a maintain all findings designation area 406 and a finding-specific maintenance designation area 407.

The medical images 310-i obtained from the case DB 102 are displayed on the medical image displaying area 401. The medical images 310-i may be configured from a plurality of cross-sectional images, and in this case, a cross-sectional position to be displayed can be changed based on a predetermined operation by the keyboard 209 or the mouse 210. Also, in a case where there is information indicating the position and range of the lesion as the medical information attached to the medical images 310-i, the medical information is clearly indicated. For example, a diagram such as a rectangle on the medical image is overlaid and displayed.

A type of the imaging finding is displayed on the finding type displaying area 402. Here, “overall shape”, “boundary”, “spicula”, sawtooth edge”, “bronchi translucency”, and the like are included in the type of the imaging finding.

The value of the imaging findings corresponding to the finding type displayed on the finding type displaying area 402 is displayed in the finding value displaying area 403. The value of the imaging findings displayed in the finding value displaying area 403 is a value obtained by the finding obtaining unit 301 based on analysis of the medical image. In an example of the user interface screen 400 of FIG. 4, it is indicated that the value of the finding type “overall shape” is “irregular shape”, the value of the finding type “boundary” is “clear”, and the value of the finding type “spicula” is “present”. Also, on the user interface screen 400, it is indicated that the value of the finding type “sawtooth edge” is “present” and the value of the finding type “bronchi translucency” is “present”.

Furthermore, each row of the finding value displaying area 403 is a pull-down menu and the values of the imaging finding can be changed. For example, when the icon (black triangular mark) on the right side at which the finding value “irregular shape” is displayed is clicked by the mouse 210, a list of contents (values) of imaging findings that can be taken by the finding type “overall shape” such as “spherical shape”, “lobed shape”, “polygonal shape”, “wedge shape”, “flat shape”, “irregular shape” is displayed as candidates. By selecting the content (value) of one imaging finding among the displayed candidates by clicking with the mouse 210, it is possible to change them to the content (value) of the imaging finding that were clicked. In other words, the first changing unit 304 changes the content of at least one finding among the plurality of findings based on an instruction (first instruction) from the input unit (the keyboard 209 or the mouse 210) with respect to the finding value displaying area 403. The display control unit (the CPU 203 and the display interface 206) causes the display 207 to display the finding value displaying area 403 in order to input an instruction (first instruction) from the input unit (the keyboard 209 or the mouse 210).

The selected state displaying area 404 displays the selected state of whether or not the imaging finding is selected by the finding selection unit 302. In the example of FIG. 4, it is indicated that the imaging findings denoted by “◯” are imaging findings selected by the finding selection unit 302, and that the selected imaging findings are “overall shape”, “spicula”, and “saw-tooth edge”.

Text of the report generated by the report generation unit 303 is displayed in the report displaying area 405. In the example of FIG. 4, it is indicated that a report saying “A node of an irregular shape is recognized in the right lung. Accompanied by spicula and a sawtooth edge” has been generated.

The maintain all findings designation area 406 is an area for designating that all findings will maintain the selected state, and in the example of FIG. 4, it is displayed in a checkbox format. The selected state maintaining unit 306 operates so that all findings will maintain the selected state when the checkbox of the maintain all findings designation area 406 is checked. The selected state maintaining unit 306 collectively sets whether or not to maintain the selected state of all the plurality of findings. In the example of FIG. 4, because the checkbox is not checked, the selected state maintaining unit 306 does not maintain the selected state of all findings.

The finding-specific maintenance designation area 407 is an area for designating that each imaging finding will maintain the selected state, and in the example of FIG. 4, it is displayed in a checkbox format for each imaging finding. The selected state maintaining unit 306 operates so that the corresponding imaging findings will maintain the selected state when the checkbox of the finding-specific maintenance designation area 407 is checked. The selected state maintaining unit 306 sets whether or not to maintain the selected state for at least one finding among the plurality of findings based on an instruction (second instruction) from an input unit such as the keyboard 209 or the mouse 210 as an operation input from the user. The selected state maintaining unit 306 sets whether or not to maintain the selected state for each of the plurality of findings. The display control unit (the CPU 203 and the display interface 206) causes the display 207 to display the finding-specific maintenance designation area 407 for inputting an instruction (second instruction) from the input unit (the keyboard 209 or the mouse 210).

In the example of FIG. 4, the selected state maintaining unit 306, regarding the finding type “overall shape” selected for the generation of the report, operates so that “overall shape” maintains the selected state, and regarding the finding type “boundary” not selected for the generation of the report, operates so that “boundary” maintains the unselected state.

### Processing Flow

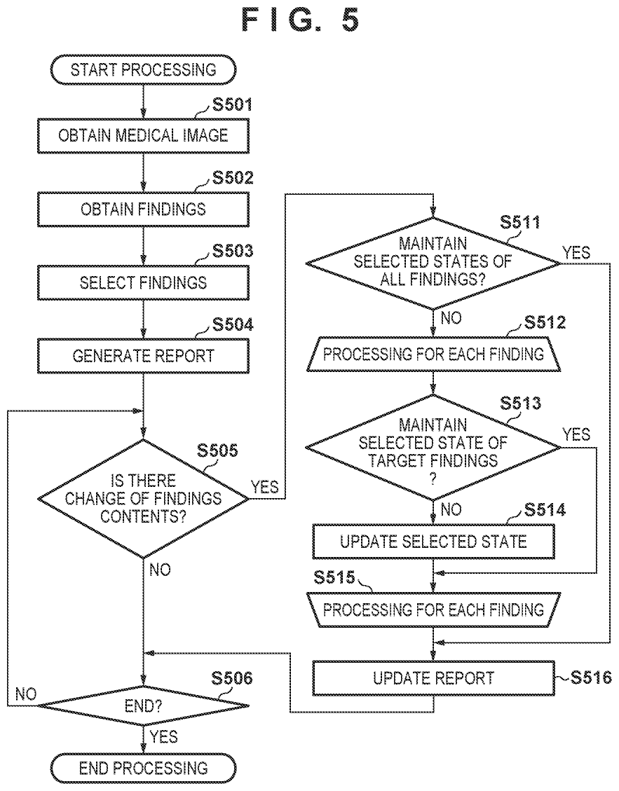

FIG. 5 is a flowchart illustrating a flow of processing of the medical information processing apparatus 101 according to the present embodiment. This processing flow is started based on an instruction by another apparatus included in the medical information processing system 10, another system, or the user after activation of the medical information processing apparatus 101. When the processing starts, a case to be processed is designated.

In step S501, the finding obtaining unit 301 obtains a medical image 310-i from the case DB 102 via the LAN 103 (network). The finding obtaining unit 301 displays an obtained medical image 310-i in the medical image displaying area 401.

In step S502, the finding obtaining unit 301 estimates imaging findings from the medical image 310-i obtained in step S501. The finding obtaining unit 301 displays the estimated imaging findings in the finding value displaying area 403.

In step S503, the finding selection unit 302 selects the imaging findings to be used in the report from the imaging findings obtained in step S502. The finding selection unit 302 displays the result of the selection in the selected state displaying area 404.

In step S504, the report generation unit 303 generates text of the report by using the imaging findings selected in step S503 and displays the generated text of the report in the report displaying area 405.

In step S505, the first changing unit 304 determines whether or not contents (values) of the imaging findings were changed by an operation in the finding value displaying area 403. In a case where the contents of the imaging findings were not changed (No in step S505), the processing advances to step S506, and in a case where the contents of the imaging findings were changed (Yes in step S505), the processing advances to step S511.

In step S506, the OS determines whether or not the present processing has ended. In a case were an end is detected (Yes in step S506), the processing ends, and in a case where an end is not detected (No in step S506), the processing returns to step S505 and the same processing is repeated.

Meanwhile, in step S511, the selected state maintaining unit 306 determines whether or not the selected state of all findings were designated to be maintained by operation in the maintain all findings designation area 406. In a case where the selected state of all findings was designated to be maintained (Yes in step S511), the processing advances to step S516, and in a case where the selected state of all findings was not designated to be maintained (No in step S511), the processing advances to step S512.

The processing from step S512 to step S515 is repeatedly performed for each of all the imaging findings obtained by the finding obtaining unit 301. Specifically, the processing is performed for “overall shape”, “boundary”, “spicula”, “sawtooth edge”, “bronchi translucency”, and the like as illustrated in FIG. 4.

In step S513, the selected state maintaining unit 306 determines whether or not the selected state of imaging findings to be processed were designated to be maintained by operation in the finding-specific maintenance designation area 407. In a case where the selected state of the imaging findings to be processed were designated to be maintained (Yes in step S513), the processing advances to step S515, and in a case where the selected state of the imaging findings to be processed were not designated to be maintained (No in step S513), the processing advances to step S514.

In step S514, the updating unit 305 updates the selected state of the imaging findings to be processed to the selected state that was selected using the imaging findings based on the change detected in step S505, and updates the display of the selected state displaying area 404.

When the processing from step S512 to step S515 ends for each of all the imaging findings, the processing advances to step S516.

In step S516, the updating unit 305 updates the report based on the content of the findings selected in accordance with the setting of whether or not to maintain the selected state set by the selected state maintaining unit 306 and the changes in the first changing unit 304. The updating unit 305 regenerates the report by using the imaging findings selected in accordance with the setting of whether or not to maintain the selected state, and updates the text of the report to be displayed in the report displaying area 405. The processing advances to step S506 and the OS determines whether or not to end the present processing.

As described above, by virtue of the present embodiment, in a case where the user requests to maintain the selected state of the imaging findings used in the report, with whether or not to mention the imaging findings in the report maintained based on the designation of maintenance of the selected state, operation is performed to generate text of the report by updating the imaging findings used in the report based on the designation of a change of the contents of imaging findings. By this, it becomes possible to provide a medical information processing technique in which it is possible for a user to set findings to be or not to be mentioned in a report as a setting of a selected state of findings.

### Variation 1 of the First Embodiment

In the first embodiment, although description regarding an example in which a chest is captured as an example of an image capturing region, limitation is not made to this example, and the medical information processing apparatus 101 may target images of parts other than the chest, such as the abdomen, mammary glands, and head. Also, an apparatus that captures a medical image is not limited to an X-ray CT apparatus, and medical images captured by an apparatus other than an X-ray CT such as a Magnetic Resonance imaging (MRI), ultrasound, or a simple X-ray may be targeted. Also, a lesion is not limited to a pulmonary nodule, and a lesion other than a pulmonary nodule such as a diffusive lung disease, a breast mass, or a hepatic mass may be targeted. Also, a diagnosis other than an image diagnosis such as a pathological diagnosis or a clinical diagnosis may be targeted. In this case, the findings may not be imaging findings but findings according to a diagnosis to be performed such as clinical findings such as an inspection, palpation, or blood test, and pathological findings can be assigned as a target. Also, the report to be generated may be text information used in documents other than an interpretation report, such as a pathological diagnosis report and a medical record.

The finding obtaining unit 301 may obtain findings by a method by other machine-learning not limited to a CNN, such as calculating an image feature amount then estimating the result by a Support Vector Machine (SVM). Also, findings may be obtained from another apparatus such as an image processing work station. Also, the user may input findings via a user interface screen. Also, findings may be extracted from text information such as text in natural language.

The finding selection unit 302 may infer a diagnostic name by a Deep Neural Network (DNN), calculate a slope of each input node for the inference result, and select the magnitude of the slope as the degree of influence. Also, supervisory data may be created by inputting all findings and outputting the selected findings, and the supervisory data may be selected by a machine-learned selector. Also, a rule base in which all findings are inputted and the selected findings are outputted may be constructed and selection may be made by the rule base.

Also, as another findings selection method, the finding selection unit 302 can statistically analyze findings specific to diagnostic names from past cases and select findings specific to diagnostic inference results (diagnostic names) of cases to be supported. Also, a doctor may empirically define in advance the findings to be selected in association with the diagnostic name, and the finding selection unit 302 may select the findings that match the definition according to the diagnostic inference result of the case to be supported.

The report generation unit 303 can also generate a report by generating sentences using a known Markov chain, Long Short-Term Memory (LSTM), or the like.

The first changing unit 304 may change values of an imaging finding by a voice input. Also, it may change content (value) of an imaging finding based on information obtained from another apparatus such as an image processing work station.

The updating unit 305 may perform updating processing at a timing at which instructions are received from the user such as a button click operation detection on the user interface screen.

The selected state maintaining unit 306 may determine a finding that will maintain a selected state based on setting information such as a registry provided by a file or the OS. Also, it may determine whether or not to maintain a selected state based on information obtained from another application or apparatus.

### Second Embodiment

The medical information processing apparatus 101 of the second embodiment adds to the medical information processing apparatus 101 of the first embodiment a function by which the user changes the selected state, and the medical information processing apparatus 101 operates so that, regarding the imaging finding for which the user changed the selected state, the selected state is maintained. Note, the system configuration of the medical information processing apparatus 101 of the present embodiment is the same as that of the first embodiment described using FIG. 1, and because the hardware configuration is the same as that of the first embodiment described using FIG. 2, description thereof is omitted.

### Functional Block

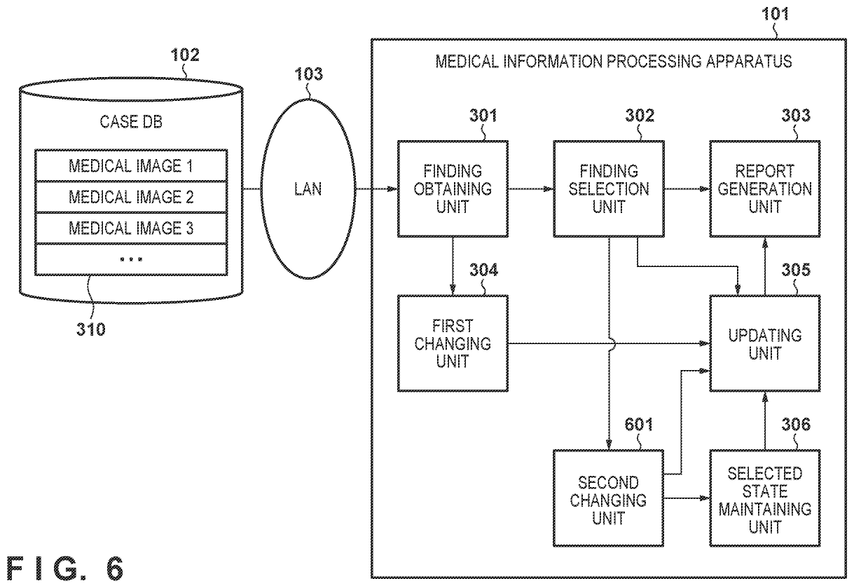

FIG. 6 is a view illustrating a functional configuration of the medical information processing apparatus 101 of the present embodiment. In FIG. 6, the medical information processing apparatus 101 of the present embodiment has a second changing unit 601 in addition to each functional block of the medical information processing apparatus 101 of the first embodiment. In addition, some operations are added to the updating unit 305 and the selected state maintaining unit 306 in accordance with the addition of the second changing unit 601.

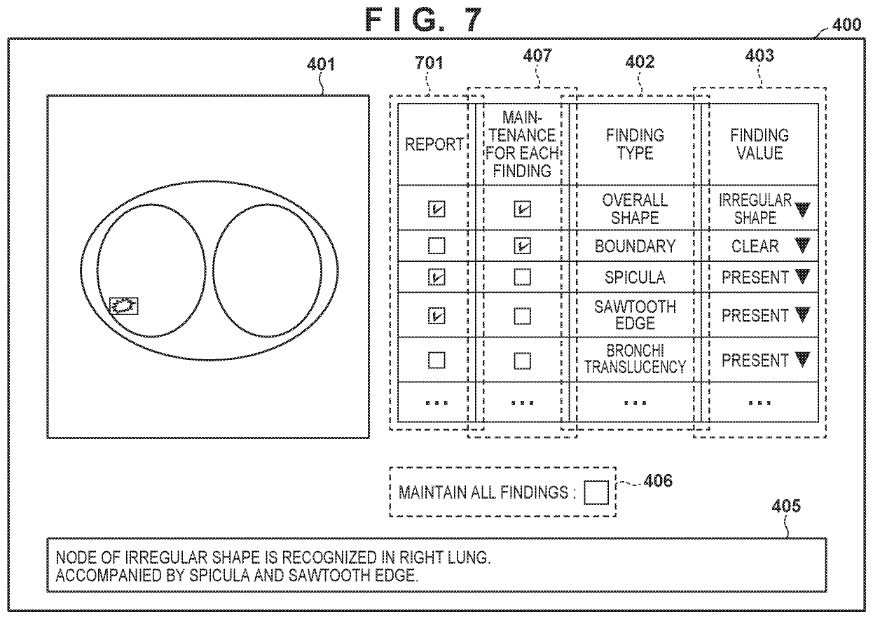

The second changing unit 601 changes the selected state for at least one finding among a plurality of findings based on an instruction (third instruction) from an input unit such as the keyboard 209 or the mouse 210 as an operation input from the user. In other words, the second changing unit 601 changes the selected state of the imaging findings selected by the finding selection unit 302 based on the user instruction. The change is performed according to an operation on the user interface screen such as a checkbox. Description regarding the user interface screen is given using FIG. 7.

The updating unit 305, in addition to the functions of the first embodiment, receives the change of the selected state by the second changing unit 601 and regenerates and updates a report based on the changed selected state. In other words, the updating unit 305 receives a change of a value of imaging findings by the first changing unit 304 and a change of a selected state by the second changing unit 601, and regenerates and updates the report by using the changed imaging findings and the unchanged imaging findings.

The selected state maintaining unit 306, in addition to the functions of the first embodiment, operates so that, regarding the imaging findings whose selected states were changed by the second changing unit 601, the selected states will be maintained. In other words, when a report will be updated by the updating unit 305, based on a designation of whether or not the imaging findings are to be used in the regeneration of the report, although the selected state maintaining unit 306 operates so as to maintain the selected states, at this time, operates so that, regarding the imaging findings whose selected states were changed by the second changing unit 601, the selected state will be maintained.

### User Interface Screen

FIG. 7 is a view illustrating an example of the user interface screen 400 of the medical information processing apparatus 101 according to the present embodiment. For the user interface screen 400 of the present embodiment, the selected state displaying area 404 (FIG. 4) of the first embodiment is changed to a selected state changing area 701.

The selected state changing area 701 displays a selected state of whether or not an imaging finding was selected by the finding selection unit 302 and also is an area for instructing a change of a selected state. The second changing unit 601, changes the selected state for at least one finding among a plurality of findings based on an instruction (third instruction) from an input unit (the keyboard 209 or the mouse 210) to the selected state changing area 701 (FIG. 7). The display control unit (the CPU 203 and the display interface 206) causes the display 207 to display the selected state changing area 701 in order to input an instruction (third instruction) from the input unit (the keyboard 209 or the mouse 210).

In the example of FIG. 7, it is displayed in the form of a check box, and a checked check box indicates a selected imaging finding, and an unchecked check box indicates an unselected imaging finding. By clicking the checkbox by the mouse 210, whether or not to check the selected state can be changed. In the example of FIG. 7, the finding types “overall shape”, “spicule”, and “sawtooth edge” are shown to be selected. The selected state maintaining unit 306 operates so that the selected state changed by the second changing unit 601 is maintained, and in the example of FIG. 7, the selected state of the selected finding types (“overall shape”, “spicula”, and “sawtooth edge”) is maintained,

### Processing Flow

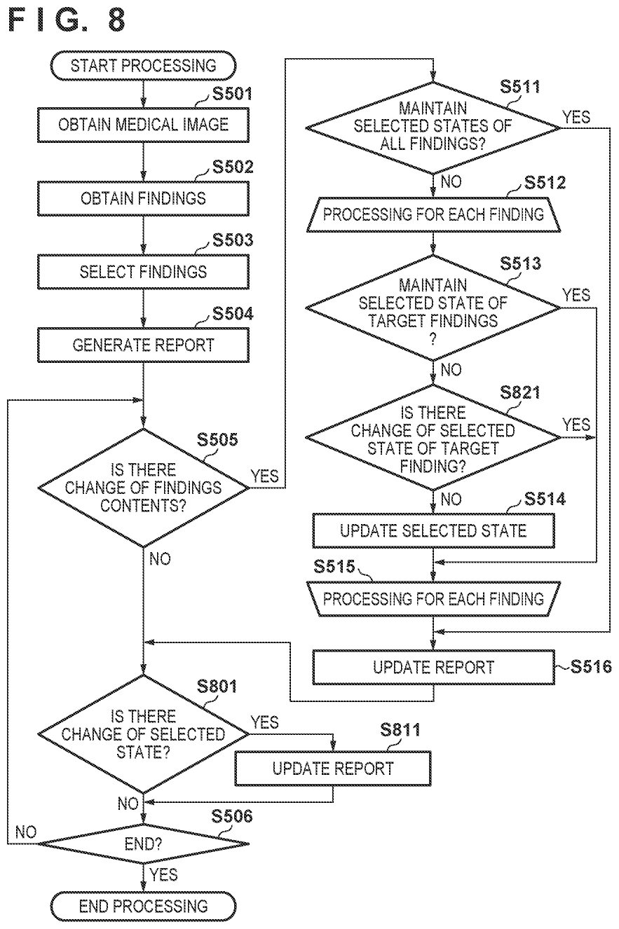

FIG. 8 is a flowchart illustrating a flow of processing of the medical information processing apparatus 101 according to the present embodiment. In a processing flow in the present embodiment, the processing in steps S801, S811 and S821 are added to the processing flow in the first embodiment, and the added processing steps are explained hereinafter.

In step S801, the second changing unit 601 determines whether or not the selected state of an imaging finding is changed by the operation in the selected state changing area 701. In a case where the selected states of the imaging findings were not changed (No in step S801), the processing advances to step S506, and in a case where the selected states of the imaging findings were changed (Yes in step S801), the processing advances to step S811.

In step S811, the updating unit 305 selects imaging findings based on the changed selected states, regenerates a report using the selected imaging findings, updates text of the report displayed on the report displaying area 405, and then advances the processing to step S506.

In step S821, the selected state maintaining unit 306 determines whether or not the imaging finding to be processed is an imaging finding whose selected state was changed. In a case where it is an imaging finding whose selected state was changed (Yes in step S821), the processing advances to step S515, and in a case where it is not an imaging finding whose selected state was changed (No in step S821), the processing advances to step S514.

As described above, by virtue of the present embodiment, in addition to the first embodiment, in a case where the user explicitly changes the selected state of the imaging findings, with whether or not to mention the imaging findings in the report maintained, operation is performed to generate text of the report by updating the imaging findings used in the report. By this, it becomes possible to provide a medical information processing technique in which it is possible for a user to set findings to be or not to be mentioned in a report as a setting of a selected state of findings. Furthermore, regarding the imaging findings whose selected state is explicitly changed, the operation for maintaining the selected state becomes unnecessary.

### Variation 1 of the Second Embodiment

The second changing unit 601 is not limited to input from the user interface screen 400 and may change the values of an imaging finding by voice input. Also, it may change the values based on information obtained from another apparatus included in the medical information processing system 10 or other systems.

The selected state maintaining unit 306 may switch whether or not to carry out the process of maintaining the selected state of the imaging findings whose selected state has been changed by the second changing unit 601 for all findings or for each finding. Also, the user may be able to designate whether or not to implement the processing to all findings, Additionally, it is also possible to designate from another apparatus included in the medical information processing system 10 or from another system or another apparatus.

By virtue of each embodiment of the present invention, it is possible for a user to set findings to be or not to be mentioned in a report, and when a report is to be updated, it becomes possible to update the report based on contents of the findings selected in accordance with a setting of selected states.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) fix performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2020-097140, filed Jun. 3, 2020, which is hereby incorporated by reference herein in its entirety.

What is claimed is:

1. A medical information processing apparatus comprising:
an obtaining unit configured to obtain a plurality of findings related to a lesion to be diagnosed;
a selection unit configured to select a finding based on a predetermined condition from the plurality of findings;
a report generation unit configured to generate, based on content of the selected finding, a report relating to the lesion;
a setting unit configured to set whether or not to maintain a selected state of the finding selected by the selection unit; and
an updating unit configured to update the report based on content of a finding selected in accordance with a setting of whether or not to maintain the selected state set by the setting unit.

2. The medical information processing apparatus according to claim 1, further comprising: a first changing unit configured to change, based on a first instruction from an input unit, content of at least one finding among the plurality of findings,
wherein the updating unit updates the report based on content of a finding selected in accordance with a setting of whether or not to maintain the selected state set by the setting unit and a change in the first changing unit.

3. The medical information processing apparatus according to claim 2, wherein, when the selection unit reselects at least one finding among the plurality of findings in accordance with the change, selects the finding according to a setting of the setting unit.

4. The medical information processing apparatus according to claim 2, wherein the setting unit sets whether or not to maintain the selected state for at least one finding among the plurality of findings based on a second instruction from the input unit.

5. The medical information processing apparatus according to claim 1, wherein the setting unit sets whether or not to maintain the selected state for each of the plurality of findings.

6. The medical information processing apparatus according to claim 1, wherein the setting unit collectively sets whether or not to maintain a selected state of all of the plurality of findings.

7. The medical information processing apparatus according to claim 4, further comprising: a second changing unit configured to change, based on a third instruction from the input unit, a selected state for at least one finding among the plurality of findings,
wherein the setting unit maintains the selected state changed by the second changing unit.

8. The medical information processing apparatus according to claim 1, wherein the report generation unit generates text of the report by applying the finding selected by the selection unit to a template.

9. The medical information processing apparatus according to claim 1, wherein the obtaining unit obtains a medical image to be diagnosed and obtains the finding by analyzing the medical image.

10. The medical information processing apparatus according to claim 9, wherein the obtaining unit, in addition to the medical image, obtains medical information including coordinate information which indicates a position and range of a lesion in a medical image and
obtains the finding by analysis using a part of the medical image as an input based on the position and range of the lesion.

11. The medical information processing apparatus according to claim 7, further comprising: a display control unit configured to display a finding value displaying area for inputting the first instruction from the input unit on a display unit.

12. The medical information processing apparatus according to claim 11, wherein the display control unit causes the display unit to display a finding-specific maintenance designation area for inputting the second instruction from the input unit.

13. The medical information processing apparatus according to claim 11, wherein the display control pit causes the display unit to display a selected state changing area for inputting the third instruction from the input unit.

14. A medical information processing system comprising:
an obtaining unit configured to obtain a plurality of findings related to a lesion to be diagnosed;
a selection unit configured to select a finding based on a predetermined condition from the plurality of findings;
a report generation unit configured to generate, based on content of the selected finding, a report relating to the lesion;
a setting unit configured to set whether or not to maintain a selected state of the finding selected by the selection unit; and
an updating unit configured to update the report generated based on content of the selected finding in accordance with a setting of whether or not to maintain the selected state set by the setting unit.

15. A medical information processing method comprising: obtaining a plurality of findings related to a lesion to be diagnosed;
selecting a finding based on a predetermined condition from the plurality of findings;
generating, based on content of the selected finding, a report relating to the lesion;
setting whether or not to maintain a selected state of the selected finding; and
updating the report based on the content of the selected finding in accordance with a setting of whether or not to maintain the selected state set by the setting.

16. A non-transitory computer-readable storage medium operable to store a program for causing a computer to function as each unit included a medical information processing apparatus according to claim 1.
